(19) United States
(12) Patent Application Publication
(10) Pub. No.: US 2011/0294686 A1
(43) Pub. Date: Dec. 1, 2011

(54) EGFR INHIBITOR THERAPY RESPONSIVENESS

(76) Inventors: Harry A. Drabkin, Charleston, SC (US); Robert M. Gemmill, Charleston, SC (US); Marileila Varella-Garcia, Greenwood Village, CO (US); Federico R. Cappuzzo, Denver, CO (US); Wan Lam, Vancouver (CA)

(21) Appl. No.: 13/063,260

(22) PCT Filed: Sep. 11, 2009
(86) PCT No.: PCT/US09/56629

§ 371 Date: Aug. 23, 2011

Related U.S. Application Data
(60) Provisional application No. 61/096,220, filed on Sep. 11, 2008.

(51) Int. Cl. C40B 30/04; C12Q 1/68; C40B 40/06

(52) U.S. Cl. 506/9; 506/16; 435/6.11

(57) ABSTRACT

Disclosed is the identification, provision and use of biomarkers predictive of sensitivity or resistance to EGFR inhibitors such as gefitinib and products and processes related thereto. Specifically, a method is described for selecting a cancer patient who is predicted to benefit from therapeutic administration of an EGFR inhibitor, an agonist thereof, or a drug having substantially similar biological activity as EGFR inhibitor. Also described is a method to identify molecules that interact with the EGFR pathway to allow or enhance responsiveness to EGFR inhibitors, as well as a plurality of polynucleotides or antibodies for the detection of the copy number or expression of genes that are indicative of sensitivity or resistance to EGFR inhibitors, an agonist thereof, or a drug having substantially similar biological activity as EGFR inhibitors. A method to identify a compound with the potential to enhance the efficacy of EGFR inhibitors is also described.

### GOVERNMENT INTEREST

This invention was made with Government support under grant number P50 CA058187 awarded by the National Institutes of Health (NIH) and the National Cancer Institute (NCI). The Government has certain rights in this invention.

### TECHNICAL FIELD

This invention generally relates to methods to screen for patients that are predicted to benefit from therapeutic administration of EGFR inhibitors, such as gefitinib, erlotinib and cetuximab, as well as methods to identify compounds that interact with the epidermal growth factor receptor (EGFR) pathway to allow or enhance responsiveness to EGFR inhibitors, and products and methods related thereto.

### BACKGROUND OF INVENTION

Lung Cancer is the leading cause of death from cancer worldwide. Chemotherapy is the mainstay of treatment for lung cancer. However, less than a third of patients with advanced stages of non-small cell lung cancer (NSCLC) respond to the best two chemotherapy drug combinations. Therefore, novel agents that target cancer-specific biological pathways are needed.

The epidermal growth factor receptor (EGFR) is one of the most appealing targets for novel therapies for cancer. EGFR plays a major role in transmitting stimuli that lead to proliferation, growth and survival of various cancer types, including, but not limited to, NSCLC. Ligand binding to the EGFR receptor leads to homo- or hetero-dimerization of EGFR with other ErbB receptors. EGFR is overexpressed in a large proportion of invasive NSCLC and in pre-malignant bronchial lesions. Activation of EGFR leads to simultaneous activation of several signaling cascades including the MAPK pathway, the protein kinase C (PKC) pathway and the PI(3)K-activated AKT pathway. EGFR signaling translated in the nucleus leads to cancer cell proliferation.

Targeted therapy against the EGFR receptor has produced response rates of 25-30% as first line treatment and 11-20% in 2^{nd} and 3^{rd} line settings (e.g., chemo-refractory advanced stage NSCLC). For example, in phase II clinical trials, 11-20% of patients with chemo-refractory advanced stage NSCLC responded to treatment with the EGFR tyrosine kinase inhibitor gefitinib (commercially available as Iressa®, ZD1839). A retrospective analysis of 140 patients responding to treatment with gefitinib revealed that the presence of BAC features (p=0.005) and being a never smoker (p=0.007) were the only independent predictors of response to gefitinib. These data suggest that EGFR inhibitor therapy is more active in BAC and in non-smokers but a need exists to determine which cancer patients will respond to EGFR inhibitor treatment.

There are no selection criteria for determining which NSCLC patients will benefit from treatment with EGFR inhibitors. Moreover, EGFR expression does not predict EGFR inhibitor sensitivity. Therefore, despite the correlation of tumor histology and smoking history with EGFR inhibitor response, it is of great importance to identify markers that influence EGFR inhibitor responsiveness, and to develop adjuvant treatments that enhance the response to treatment with these agents. To accomplish this goal, there is a need in the art to define genetic or protein markers indicative of responsiveness to EGFR inhibitors. This invention satisfies these needs.

### SUMMARY OF INVENTION

One embodiment of the present invention relates to a method to select a cancer patient who is predicted to benefit from therapeutic administration of an EGFR inhibitor, an agonist thereof, or a drug having substantially similar biological activity as EGFR inhibitor The method includes the steps of: (a) providing a sample of tumor cells from a patient to be tested; (b) detecting in the sample the copy number of genes chosen from a panel of genes whose copy number has been correlated with sensitivity to an EGFR inhibitor; (c) detecting in the sample the copy number of genes chosen from a panel of genes whose copy number has been correlated with resistance to an EGFR inhibitor; (d) comparing the level of copy number of the genes detected in the patient sample to the copy number of the genes that have been correlated with sensitivity to the EGFR inhibitor; (e) comparing the copy number of the genes detected in the patient sample to the copy number of the genes that have been correlated with resistance to the EGFR inhibitor; and (f) selecting a patient as predicted to benefit from therapeutic administration of the EGFR inhibitor, if the copy number of the genes in the patient's tumor cells is statistically more similar to the copy number of the genes that have been correlated with sensitivity to the EGFR inhibitor than to resistance to the EGFR inhibitor.

In one aspect, the panel of genes is identified by a method that includes the steps of: (a) providing a sample of tumor cells that are sensitive to treatment with the EGFR inhibitor; (b) providing a sample of tumor cells that are resistant to treatment with the EGFR inhibitor; (c) detecting the copy number of at least one gene in the EGFR inhibitor-sensitive cells as compared to the copy number of at least one gene in the EGFR inhibitor-resistant cells; and (d) identifying genes having a copy number in EGFR inhibitor-sensitive cells that are statistically significantly different than the copy number of the genes in EGFR inhibitor-resistant cells, as potentially being a molecule that interacts with the EGFR pathway to enhance responsiveness to EGFR inhibitors.

In another aspect, the EGFR inhibitor is gefitinib, erlotinib and cetuximab.

In yet another aspect, the genes chosen from the panel of genes are MYC and EIF3H genes.

In one aspect of the invention, steps (b) and (c) include detecting in the sample the copy number of at least one of the MYC and EIF3H genes. Steps (d) and (e) include comparing the copy number of the genes detected in the patient sample to a copy number of the genes that have been correlated with sensitivity to an EGFR inhibitor such as gefitinib and to resistance of an EGFR inhibitor such as gefitinib. Step (f) includes selecting the patient as being predicted to benefit from therapeutic administration of an EGFR inhibitor such as gefitinib, an agonist thereof, and a drug having substantially similar biological activity as an EGFR inhibitor like gefitinib, if the copy number of the genes in the patient's tumor cells is statistically more similar to the copy number of the genes that have been correlated with sensitivity to an EGFR inhibitor such as gefitinib than to resistance to an EGFR inhibitor like gefitinib.

In another aspect, the steps of detecting include detecting the copy number of at least two genes in steps (b) and (c). In one aspect, the steps of detecting include detecting the copy number of substantially all of the genes in the panel of genes. In yet another aspect, the steps of detecting include detecting the copy number of substantially all of the genes in steps (b) and (c).

In one aspect of this method, the copy number of the genes is detected by measuring the hybridization of nucleic acid probes that hybridize specifically with the MYC or EIF3H gene sequences. In preferred embodiments, these measurements may take the form of fluorescent in situ hybridization, comparative genomic hybridization, array comparative genomic hybridization and SNP genotyping. In another aspect, expression of the MYC or EIF3H genes is detected by detecting hybridization of at least a portion of the gene, or a transcript thereof, to a nucleic acid molecule comprising a portion of the gene or a transcript thereof in a nucleic acid array. In another aspect, expression of the gene is detected by detecting the production of a protein encoded by the gene.

In one aspect of this method, copy number of the genes is detected by measuring amounts of transcripts of the genes in the tumor cells. In another aspect, the copy number of the genes is detected by hybridization of one of a portion of the gene and a transcript thereof, to a nucleic acid molecule comprising one of a portion of the gene and a transcript thereof, conjugated to a detectable marker. In yet another aspect, copy number of the genes is detected by Fluorescent in situ hybridization (FISH). In another aspect, the copy number of the genes is detected by detecting the production of proteins encoded by the genes. In one aspect, the method includes comparing the copy number of the genes to the copy number of the genes in a cell from a non-cancerous cell of the same type. In another aspect, the method includes comparing the copy number of the genes to the copy number of the genes in an autologous, non-cancerous cell from the patient. In another aspect the method includes comparing the copy number of the genes to the copy number of the genes in a control cell that is resistant to an EGFR inhibitor. In yet another aspect, the method includes comparing the copy number of the genes to the copy number of the genes in a control cell that are sensitive to an EGFR inhibitor. In one aspect the control copy number of the genes that have been correlated with sensitivity to the EGFR inhibitor have been predetermined and in another aspect, the control copy number of the genes that have been correlated with resistance to the EGFR inhibitor have been predetermined.

In one aspect, the genes include the MYC gene and the EIF3H gene. In this aspect, amplification (through increased copy number, over expression or enhanced activity of the protein product) of the MYC gene is associated with improved response to anti-EGFR cancer treatment and the identification of a patient predicted to have an improved outcome of cancer treatment following administration of anti-EGFR therapy. In a similar aspect, amplification (through increased copy number, over expression or enhanced activity of the protein product) of the EIF3H gene is associated with improved response to anti-EGFR cancer treatment and the identification of a patient predicted to have an improved outcome of cancer treatment following administration of anti-EGFR therapy. Amplification of both the MYC gene and the EIF3H gene is associated with improved response to anti-EGFR cancer treatment and the identification of a patient predicted to have an improved outcome of cancer treatment following administration of anti-EGFR therapy.

Another embodiment of the present invention relates to a method to select a cancer patient who is predicted to benefit from therapeutic administration of an EGFR inhibitor, an agonist thereof, and a drug having substantially similar biological activity as an EGFR inhibitor. The method includes the steps of: (a) providing a sample of tumor cells from a patient to be tested; (b) detecting in the sample the expression of genes chosen from a panel of genes whose expression has been correlated with sensitivity to an EGFR inhibitor; (c) detecting in the sample the expression of genes chosen from a panel of genes whose expression has been correlated with resistance to an EGFR inhibitor; (d) comparing the level of expression of the genes detected in the patient sample to a level of expression of the genes that have been correlated with sensitivity and resistance to the EGFR inhibitor; and (e) selecting the patient as being predicted to benefit from therapeutic administration of the EGFR inhibitor, if the expression of the genes in the patient's tumor cells is statistically more similar to the expression levels of the genes that have been correlated with sensitivity to the EGFR inhibitor than to resistance to the EGFR inhibitor.

In one aspect, the panel of genes in steps (b) and (c) are identified by a method that includes the steps of: (a) providing a sample of cells that are sensitive to treatment with the EGFR inhibitor; (b) providing a sample of cells that are resistant to treatment with the EGFR inhibitor; (c) detecting the expression of at least one gene in the EGFR inhibitor-sensitive cells as compared to the level of expression of at least one gene in the EGFR inhibitor-resistant cells; and (d) identifying genes having a level of expression in EGFR inhibitor-sensitive cells that is statistically significantly different than the level of expression of the genes in EGFR inhibitor-resistant cells, as potentially being a molecule that interacts with the EGFR pathway to enhance responsiveness to EGFR inhibitors. In another aspect the EGFR inhibitor is gefitinib, erlotinib and cetuximab and in yet another aspect the genes chosen from the panel are MYC and EIF3H genes.

Therefore in one aspect of the invention, steps (b) and (c) include detecting in the sample the expression of at least one of MYC and EIF3H genes. Step (d) includes comparing the level of expression of the genes detected in the patient sample to a level of expression of the genes that have been correlated with sensitivity and resistance to gefitinib. Step (e) includes selecting the patient as being predicted to benefit from therapeutic administration of an EGFR inhibitor such as gefitinib, an agonist thereof, and a drug having substantially similar biological activity as an EGFR inhibitor like gefitinib, if the expression of the genes in the patient's tumor cells is statistically more similar to the expression levels of the genes that have been correlated with sensitivity to the EGFR inhibitor like gefitinib than to resistance to the EGFR inhibitor such as gefitinib.

In another aspect the steps of detecting include detecting expression of at least two genes in steps (b) and (c). In one aspect, the steps of detecting include detecting expression of substantially all of the genes in the panel of genes. In yet another aspect, the steps of detecting include detecting substantially all of the genes in steps (b) and (c).

In one aspect of this method, the expression of the genes is detected by measuring amounts of transcripts of the genes in the tumor cells. In yet another aspect, expression of the genes is detected by detecting hybridization of at least a portion of the gene or a transcript thereof, to a nucleic acid molecule comprising a portion of the gene and a transcript thereof in a nucleic acid array. In another aspect the expression of the genes is detected by detecting the production of proteins encoded by the genes.

Another aspect, the method includes comparing the expression of the genes to the expression of the genes in a cell from a non-cancerous cell of the same type. In yet another aspect, the method includes comparing the expression of the genes to the expression of the genes in an autologous, non-cancerous cell from the patient. In one aspect, the method includes comparing the expression of the genes to the expression of the genes in a control cell that is resistant to the EGFR inhibitor. In yet another aspect, the method includes comparing the expression of the genes to the expression of the genes in a control cell that is sensitive to the EGFR inhibitor. In another aspect the control expression levels of the genes that have been correlated with sensitivity to the EGFR inhibitor have been predetermined and the control expression levels of the genes that have been correlated with resistance to the EGFR inhibitor have been predetermined

Another embodiment of the present invention relates to a method to identify molecules that interact with the EGFR pathway to enhance responsiveness to EGFR inhibitors. The method includes the steps of (a) providing a sample of cells that are sensitive to treatment with an EGFR inhibitor; (b) providing a sample of cells that are resistant to treatment with an EGFR inhibitor; (c) detecting the copy number of at least one gene in the EGFR inhibitor-sensitive cells as compared to the copy number of at least one gene in the EGFR inhibitor-resistant cells; and (d) identifying genes having a copy number in EGFR inhibitor-sensitive cells that are statistically significantly different than the copy number of the genes in EGFR inhibitor-resistant cells, as molecules that interact with the EGFR pathway to enhance responsiveness to EGFR inhibitors.

Another embodiment of the present invention relates to a plurality of polynucleotides for the detection of the copy number of genes such as EGFR inhibitor-sensitive genes, EGFR inhibitor-resistant genes, agonists thereof, and drugs having substantially similar biological activity as EGFR inhibitors. The plurality of polynucleotides consists of at least two polynucleotides, wherein each polynucleotide is at least 5 nucleotides in length, and wherein each polynucleotide is complementary to a genomic sequence, an RNA transcript, and nucleotides derived therefrom, of a gene that has a gene copy number that is different in EGFR inhibitor-sensitive tumor cells as compared to EGFR inhibitor-resistant cells. In one aspect, each polynucleotide is complementary to a genomic sequence, an RNA transcript, a polynucleotide derived therefrom, a MYC gene and an EIF3H gene. In another aspect, the plurality of polynucleotides are complementary to a genomic sequence, an RNA transcript, and a nucleotide derived therefrom, of both MYC and EIF3H genes. In yet another aspect, the polynucleotides are probes. In one aspect the polynucleotide probes are immobilized on a substrate. In another aspect the polynucleotide probes are hybridizable array elements in a microarray. In yet another aspect the polynucleotide probes are conjugated to detectable markers.

Yet another embodiment of the present invention relates to a plurality of antibodies, antigen binding fragments thereof, or antigen binding peptides, for the detection of the copy number or expression of genes that are indicative of EGFR inhibitor sensitive genes, EGFR inhibitor-resistant genes, an agonist thereof, and a drug having substantially similar biological activity as EGFR inhibitor. The plurality of antibodies, antigen binding fragments thereof, or antigen binding peptides consists of at least two antibodies, antigen binding fragments thereof, or antigen binding peptides, each of which selectively binds to a protein encoded by a gene comprising, or expressing a transcript comprising, a nucleic acid sequence of the MYC or EIF3H genes.

Another embodiment of the present invention relates to a method to identify a compound that enhances the efficacy of EGFR inhibitors. The method includes the steps of: (a) contacting a test compound with a cell that expresses at least one of the MYC and EIF3H genes; (b) identifying compounds that increase the expression or activity of the genes in (a), or the proteins encoded thereby that are correlated with sensitivity to an EGFR inhibitor; and compounds that decrease the expression or activity of genes in (a), or the proteins encoded thereby, that are correlated with resistance to an EGFR inhibitor. The compounds are identified as having the potential to enhance the efficacy of treatment with EGFR inhibitors.

Another aspect of the present invention is a method to treat a patient with a cancer, including administering to the patient a therapeutic composition comprising a compound identified by the method described above.

Yet another embodiment of the present invention relates to a method to treat a patient with a cancer, including administering to the patient a therapeutic composition including a compound that upregulates or down-regulates the expression of MYC and EIF3H genes in the tumor cells of the patient.

Another embodiment of the present invention is the use of a compound that down-regulates or upregulates the expression of the MYC and EIF3H genes in the preparation of a medicament for the treatment of a cancer in a patient in need of such treatment. In one aspect, the cancer is lung cancer.

### DESCRIPTION OF EMBODIMENTS

The present invention generally relates to the identification, provision and use of biomarkers that predict sensitivity or resistance to EGFR inhibitors, and products and processes related thereto. Specifically, the present inventors have used NSCLC cell lines with varying sensitivity to EGFR inhibitors, to define the biomarkers described herein. In order to identify markers that could be used for selection of cancer patients who will respond to EGFR inhibitor treatment, the inventors undertook preclinical in vitro studies using lung tumor specimens from 54 lung cancer patients. In the 54 lung tumors tested, 10 had MYC amplification. Human chromosome 8 often suffers genetic damage in lung cancer, including amplification of the MYC oncogene at 8q24.21. MYC is a negative prognostic factor and MYC amplification seems to increase sensitivity to trastuzumab (Herceptin™), a monoclonal antibody against HER2, a member of the EGFR family. In addition, all of those samples were also amplified for eukaryotic translation initiation factor 3, subunit H (EIF3H). The gene for EIF3H is also located within 8q24, is amplified in cancer, but data on EIF3H in lung cancer are lacking. Approximately, 50% of the specimens had increased gene copy numbers (>2.8 copies per cell) for both genes. In addition, as these patients had been treated with an EGFR inhibitor, it was possible to perform a statistical analysis for correlation between gene amplification and clinical outcome. This analysis revealed that high copy numbers of the EIF3H and MYC genes identified a patient subset with better outcome to anti-EGFR therapy: they have significantly higher response rate (p=0.03), longer time to progression (p=0.014) and longer survival (p=0.024). These are unexpected findings because amplification of either EIF3H or MYC has been associated with a worse prognosis in other tumor types.

Thus, one embodiment of the invention is the identification of tumors with higher copy number or expression of EIF3H and MYC that are more likely to respond to anti-EGFR therapy including EGFR inhibitor drugs, such as erlotinib, gefitinib and cetuximab. A similar embodiment of the invention is the identification of cancer patients that have tumors with higher copy number or expression of EIF3H and MYC that are more likely to respond to anti-EGFR therapy including EGFR inhibitor drugs, such as erlotinib, gefitinib and cetuximab. In one preferred aspect of the invention, the identification is conducted by the application of Fluorescence In Situ Hybridization (FISH) technology to assess the copy number for EIF3H and MYC in a tumor.

In addition, the present invention will also be useful for the validation in other studies of the clinical significance of the specific biomarkers described herein, as well as the identification of preferred biomarker profiles and targets for the design of novel therapeutic products and strategies. The MYC and EIF3H biomarkers described herein are particularly useful in clinical practice to select the patients who will benefit most from EGFR inhibitor treatment and in specific embodiments, from EGFR inhibitor treatment.

The present inventors have shown the MYC and EIF3H gene biomarkers to correlate to patients that displayed a better response to EGFR inhibitor treatment compared with a similar group of patients that showed a less favorable outcome following EGFR inhibitor treatment as described in detail in the Examples. These data indicate that the gene amplification or over-expression of MYC and/or EIF3H may predict resistance to EGFR tyrosine kinase inhibitors and modulation of the regulation of MYC and/or EIF3H expression are expected to enhance the activity of EGFR inhibitors in tumors.

Finally, the present invention also relates to protein profiles that can discriminate between EGFR inhibitor-sensitive and resistant tumors.

Using the gene expression profiles of MYC and/or EIF3H for EGFR inhibitor-sensitive and resistant cells, one can effectively and efficiently screen patients/human tumors for a level of sensitivity or resistance to an EGFR inhibitor or drugs having similar activities or EGFR inhibitor agonists and other derivatives. The results allow for the identification of tumors/patients that are likely to benefit from administration of the drug and therefore, the genes are used to enhance the ability of the clinician to develop prognosis and treatment protocols for the individual patient. In addition, MYC and EIF3H genes can be used in assays to identify therapeutic reagents useful for regulating the expression or activity of the target in a manner that improves sensitivity of a cancer to an EGFR inhibitor or analogs thereof. Given the knowledge of these genes, one of skill in the art can produce novel combinations of polynucleotides and/or antibodies and/or peptides for use in the various assays, diagnostic and/or therapeutic approaches described herein.

Various definitions and aspects of the invention will be described below, but the invention is not limited to any specific embodiments that may be used for illustrative or exemplary purposes.

According to the present invention, in general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

According to the present invention, a “downstream gene” or “endpoint gene” is any gene, the expression of which is regulated (up or down) within an EGFR inhibitor sensitive or resistant cell. Selected sets of one or two of the biomarker genes of this invention can be used as end-points for rapid screening of patient cells for sensitivity or resistance to EGFR inhibitors and for the other methods as described herein, including the identification of novel targets for the development of new cancer therapeutics.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes to one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

An “agonist” can be any compound which is capable of mimicking, duplicating or approximating the biological activity of a naturally occurring or specified protein, for example, by associating with (e.g., binding to) or activating a protein (e.g., a receptor) to which the natural protein binds, so that activity that would be produced with the natural protein is stimulated, induced, increased, or enhanced. For example, an agonist can include, but is not limited to, a protein, compound or an antibody that selectively binds to and activates or increases the activation of a receptor bound by the natural protein, other homologues of the natural protein, and any suitable product of drug design that is characterized by its ability to agonize (e.g., stimulate, induce, increase, enhance) the biological activity of a naturally occurring protein.

An “antagonist” refers to any compound or agent which is capable of acting in a manner that is antagonistic to (e.g., against, a reversal of, contrary to) the action of the natural agonist, for example by interacting with another protein or molecule in a manner that the biological activity of the naturally occurring protein or agonist is decreased (e.g., reduced, inhibited, blocked). Such a compound can include, but is not limited to, an antibody that selectively binds to and blocks access to a protein by its natural ligand, or reduces or inhibits the activity of a protein, a product of drug design that blocks the protein or reduces the biological activity of the protein, an anti-sense nucleic acid molecule that binds to a nucleic acid molecule encoding the protein and prevents expression of the protein, a ribozyme that binds to the RNA and prevents expression of the protein, RNAi, an aptamer, and a soluble protein, which competes with a natural receptor or ligand.

Agonists and antagonists that are products of drug design can be produced using various methods known in the art. Various methods of drug design, useful to design mimetics or other compounds useful in the present invention are disclosed in Maulik et al., 1997, Molecular Biotechnology: Therapeutic Applications and Strategies, Wiley-Liss, Inc., which is incorporated herein by reference in its entirety. An agonist or antagonist can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, natural or synthetic steroidal compounds, carbohydrates and/or natural or synthetic organic and non-steroidal molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

As used herein, the term “mimetic” is used to refer to any natural or synthetic compound, peptide, oligonucleotide, carbohydrate and/or natural or synthetic organic molecule that is able to mimic the biological action of a naturally occurring or known synthetic compound.

As used herein, the term “putative regulatory compound” or “putative regulatory ligand” refers to compounds having an unknown regulatory activity, at least with respect to the ability of such compounds to regulate the expression or biological activity of a gene or protein encoded thereby, or to regulate sensitivity or resistance to an EGFR inhibitor as encompassed by the present invention.

In accordance with the present invention, an isolated polynucleotide, which phrase can be used interchangeably with “an isolated nucleic acid molecule”, is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. Polynucleotides useful in the plurality of polynucleotides of the present invention (described below) are typically a portion of a gene or transcript thereof of the present invention that is suitable for use, for example, as a hybridization probe or PCR primer for the identification of a full-length gene, a transcript thereof, or a polynucleotide derived from the gene or transcript (e.g., cDNA), in a given sample (e.g., a cell sample). An isolated nucleic acid molecule can include a gene or a portion of a gene (e.g., the regulatory region or promoter), for example, to produce a reporter construct according to the present invention. An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecules can include DNA, RNA (e.g., mRNA), or derivatives of either DNA or RNA (e.g., cDNA). Although the phrase “nucleic acid molecule” or “polynucleotide” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on the biological activity of the protein as described herein. Protein homologues (e.g., proteins encoded by nucleic acid homologues) have been discussed in detail above.

The minimum size of a nucleic acid molecule or polynucleotide of the present invention is a size sufficient to encode a protein having a desired biological activity, sufficient to form a probe or oligonucleotide primer that is capable of forming a stable hybrid with the complementary sequence of a nucleic acid molecule encoding the natural protein (e.g., under moderate, high or very high stringency conditions), or to otherwise be used as a target in an assay or in any therapeutic method discussed herein. If the polynucleotide is an oligonucleotide probe or primer, the size of the polynucleotide can be dependent on nucleic acid composition and percent homology or identity between the nucleic acid molecule and a complementary sequence as well as upon hybridization conditions per se (e.g., temperature, salt concentration, and formamide concentration). The minimum size of a polynucleotide that is used as an oligonucleotide probe or primer is at least about 5 nucleotides in length, and preferably ranges from about 5 to about 50 or about 500 nucleotides, including any length in between, in whole number increments (i.e., 5, 6, 7, 8, 9, 10, . . . 33, 34, . . . 256, 257, . . . 500), and more preferably from about 10 to about 40 nucleotides, and most preferably from about 15 to about 40 nucleotides in length. In one aspect, the oligonucleotide primer or probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a portion of a protein-encoding sequence or a nucleic acid sequence encoding a full-length protein.

An isolated protein, according to the present invention, is a protein (including a peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. An isolated protein useful as an antagonist or agonist according to the present invention can be isolated from its natural source, produced recombinantly or produced synthetically. Smaller peptides useful as regulatory peptides are typically produced synthetically by methods well known to those of skill in the art.

According to the present invention, the phrase “selectively binds to” refers to the ability of an antibody, antigen binding fragment or binding partner (antigen binding peptide) to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

In some embodiments of the present invention, a compound is contacted with one or more nucleic acids or proteins. Such methods can include cell-based assays, or non-cell-based assay. In one embodiment, a target gene is expressed by a cell (i.e., a cell-based assay). In one embodiment, the conditions under which a cell expressing a target is contacted with a putative regulatory compound, such as by mixing, are conditions in which the expression or biological activity of the target (gene or protein encoded thereby) is not stimulated (activated) if essentially no regulatory compound is present. For example, such conditions include normal culture conditions in the absence of a known activating compound or other equivalent stimulus. The putative regulatory compound is then contacted with the cell. In this embodiment, the step of detecting is designed to indicate whether the putative regulatory compound alters the expression and/or biological activity of the gene or protein target as compared to in the absence of the putative regulatory compound (i.e., the background level).

In accordance with the present invention, a cell-based assay as described herein is conducted under conditions which are effective to screen for regulatory compounds or to profile gene expression as described in the methods of the present invention. Effective conditions include, but are not limited to, appropriate media, temperature, pH and oxygen conditions that permit the growth of the cell that expresses the receptor. An appropriate, or effective, medium is typically a solid or liquid medium comprising growth factors and assimilable carbon, nitrogen and phosphate sources, as well as appropriate salts, minerals, metals and other nutrients, such as vitamins. Culturing is carried out at a temperature, pH and oxygen content appropriate for the cell. Such culturing conditions are within the expertise of one of ordinary skill in the art.

Cells that are useful in the cell-based assays of the present invention include any cell that expresses a gene that is to be investigated as a target, or in the diagnostic assays described herein, any cell that is isolated from a patient, including normal or malignant (tumor) cells.

According to the present invention, the method includes the step of detecting the expression of at least one, and preferably more than one, of the genes that are regulated differently in EGFR inhibitor-sensitive versus EGFR inhibitor-resistant cells, and particularly, of the genes that have now been shown to be regulated differently in an EGFR inhibitor-sensitive versus an EGFR inhibitor-resistant cells, by the present inventors. As used herein, the term “expression”, when used in connection with detecting the expression of a gene, can refer to detecting transcription of the gene and/or to detecting translation of the gene. To detect expression of a gene refers to the act of actively determining whether a gene is expressed or not. This can include determining whether the gene expression is upregulated as compared to a control, downregulated as compared to a control, or unchanged as compared to a control. Therefore, the step of detecting expression does not require that expression of the gene actually is upregulated or downregulated, but rather, can also include detecting that the expression of the gene has not changed (i.e., detecting no expression of the gene or no change in expression of the gene).

In another embodiment of the invention, detecting in the sample the copy number of one or more genes chosen from a panel of genes whose expression has been correlated with sensitivity or resistance to an EGFR inhibitor. For example, EGFR inhibitor sensitivity is identified by a method comprising: (a) providing a sample of cells that are sensitive or resistant to treatment with the EGFR inhibitor; (b) detecting the copy number of at least one of MYC and EIF3H genes in the cells as compared to the copy number of the gene or genes in EGFR inhibitor-resistant cells; and (c) identifying a sample of cells having a copy number of MYC and EIF3H genes that is statisitically significantly similar to cells having an EGFR inhibitor-sensitivity or that have a statistically significantly different copy number of MYC and EIF3H genes present in EGFR inhibitor-resistant cells, as being responsive to EGFR inhibitors. Therefore, although many of the embodiments below are discussed in terms an EGFR inhibitor, it is to be understood that the methods of the invention can be extended to therapeutic agents that are similar in structure and/or function to an EGFR inhibitor, including agonists of an EGFR inhibitor.

The first steps of the method to select a cancer patient that is predicted to benefit from therapeutic administration of an EGFR inhibitor, an agonist thereof, or a drug having substantially similar biological activity as EGFR inhibitor of the present invention, includes providing a patient sample (also called a test sample) and detecting in the sample the expression of a gene or genes. Suitable methods of obtaining a patient sample are known to a person of skill in the art. A patient sample can include any bodily fluid or tissue from a patient that may contain tumor cells or proteins of tumor cells. More specifically, according to the present invention, the term “test sample” or “patient sample” can be used generally to refer to a sample of any type which contains cells or products that have been secreted from cells to be evaluated by the present method, including but not limited to, a sample of isolated cells, a tissue sample and/or a bodily fluid sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells that are preferably evaluated. Cells can be obtained, for example, by scraping of a tissue, processing of a tissue sample to release individual cells, or isolation from a bodily fluid.

A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch. A bodily fluid sample, like the tissue sample, contains the cells to be evaluated for marker expression or biological activity and/or may contain a soluble biomarker that is secreted by cells, and is a fluid obtained by any method suitable for the particular bodily fluid to be sampled. Bodily fluids suitable for sampling include, but are not limited to, blood, mucous, seminal fluid, saliva, breast milk, bile and urine.

In general, the sample type (i.e., cell, tissue or bodily fluid) is selected based on the accessibility and structure of the organ or tissue to be evaluated for tumor cell growth and/or on what type of cancer is to be evaluated. For example, if the organ/tissue to be evaluated is the breast, the sample can be a sample of epithelial cells from a biopsy (i.e., a cell sample) or a breast tissue sample from a biopsy (a tissue sample). The sample that is most useful in the present invention will be cells, tissues or bodily fluids isolated from a patient by a biopsy or surgery or routine laboratory fluid collection.

Once a sample is obtained from the patient, the sample is evaluated for the detection of the expression of the gene or genes that have been correlated with sensitivity or resistance to an EGFR inhibitor. For example, as discussed above, one or more of MYC and EIF3H genes are useful for detection in the present method.

In one aspect of the method of the present invention, the step of detecting can include the detection of copy number of one or more of the MYC and EIF3H genes. Copy number of these genes may be measured by any of a variety of known methods in the art. These methods may include, but are not limited to cytogenetic techniques well known in the art including: fluorescent in situ hybridization (FISH), comparative genomic hybridization (CGH) or chromosomal microarray analysis (CMA; also Array comparative genomic hybridization, Microarray-based comparative genomic hybridization, array CGH, a-CGH, or aCGH) or large-scale SNP genotyping.

In another aspect of the method of the present invention, the step of detecting can include the detection of expression of one or more of the MYC and EIF3H genes. Expression of transcripts and/or proteins is measured by any of a variety of known methods in the art. For RNA expression, methods include but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene.

Methods to measure protein expression levels generally include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry, as well as assays based on a property of the protein including but not limited to enzymatic activity or interaction with other protein partners. Binding assays are also well known in the art. For example, a BIAcore machine can be used to determine the binding constant of a complex between two proteins. The dissociation constant for the complex can be determined by monitoring changes in the refractive index with respect to time as buffer is passed over the chip (O'Shannessy et al. Anal. Biochem. 212:457 (1993); Schuster et al., Nature 365:343 (1993)). Other suitable assays for measuring the binding of one protein to another include, for example, immunoassays such as enzyme linked immunoabsorbent assays (ELISA) and radioimmunoassays (RIA); or determination of binding by monitoring the change in the spectroscopic or optical properties of the proteins through fluorescence, UV absorption, circular dichroism, or nuclear magnetic resonance (NMR). Nucleic acid arrays are particularly useful for detecting the expression of the MYC and EIF3H genes of the present invention. The production and application of high-density arrays in gene expression monitoring have been disclosed previously in, for example, WO 97/10365; WO 92/10588; U.S. Pat. No. 6,040,138; U.S. Pat. No. 5,445,934; or WO95/35505, all of which are incorporated herein by reference in their entireties. Also for examples of arrays, see Hacia et al. (1996) Nature Genetics 14:441-447; Lockhart et al. (1996) Nature Biotechnol. 14:1675-1680; and De Risi et al. (1996) Nature Genetics 14:457-460. In general, in an array, an oligonucleotide, a cDNA, or genomic DNA, that is a portion of a known gene occupies a known location on a substrate. A nucleic acid target sample is hybridized with an array of such oligonucleotides and then the amount of target nucleic acids hybridized to each probe in the array is quantified. One preferred quantifying method is to use confocal microscope and fluorescent labels. The Affymetrix GeneChip™ Array system (Affymetrix, Santa Clara, Calif.) and the Atlas™ Human cDNA Expression Array system are particularly suitable for quantifying the hybridization; however, it will be apparent to those of skill in the art that any similar systems or other effectively equivalent detection methods can also be used. In a particularly preferred embodiment, one can use the knowledge of the genes described herein to design novel arrays of polynucleotides, cDNAs or genomic DNAs for screening methods described herein. Such novel pluralities of polynucleotides are contemplated to be a part of the present invention and are described in detail below.

Suitable nucleic acid samples for screening an array contain transcripts of interest or nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like. Preferably, the nucleic acids for screening are obtained from a homogenate of cells or tissues or other biological samples. Preferably, such sample is a total RNA preparation of a biological sample. More preferably in some embodiments, such a nucleic acid sample is the total mRNA isolated from a biological sample. Biological samples may be of any biological tissue or fluid or cells from any organism. Frequently the sample will be a “clinical sample” which is a sample derived from a patient, such as a tumor sample from a patient. Typical clinical samples include, but are not limited to, sputum, blood, blood cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

In one embodiment, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids to achieve quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high-density array may then include probes specific to the internal standard for quantification of the amplified nucleic acid. Other suitable amplification methods include, but are not limited to polymerase chain reaction (PCR) Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al, Proc. Nat. Acad. Sci. USA, 87: 1874 (1990)).

Nucleic acid hybridization simply involves contacting a probe and target nucleic acid under conditions where the probe and its complementary target can form stable hybrid duplexes through complementary base pairing. As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid. Nucleic acids that do not form hybrid duplexes are washed away from the hybridized nucleic acids and the hybridized nucleic acids can then be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

High stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). One of skill in the art can use the formulae in Meinkoth et al., 1984, Anal. Biochem. 138, 267-284 to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6× SSC (0.9 M Na⁺) at a temperature of between about 20° C. and about 35° C., more preferably, between about 28° C. and about 40° C., and even more preferably, between about 35° C. and about 45° C. In particular embodiments, stringent hybridization conditions for DNA:RNA hybrids include hybridization at an ionic strength of 6× SSC (0.9 M Na⁺) at a temperature of between about 30° C. and about 45° C., more preferably, between about 38° C. and about 50° C., and even more preferably, between about 45° C. and about 55° C. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62.

The hybridized nucleic acids are detected by detecting one or more labels attached to the sample nucleic acids. The labels may be incorporated by any of a number of means well known to those of skill in the art. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., DYNABEADS™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The term “quantifying” or “quantitating” when used in the context of quantifying copy number or transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

In one aspect of the present method, in vitro cell based assays may be designed to screen for compounds that affect the regulation of the MYC and/or EIF3H genes at either the transcriptional or translational level. One, two or more promoters of the genes of this invention can be used to screen unknown compounds for activity on a given target. Promoters of the selected genes can be linked to any of several reporters (including but not limited to chloramphenicol acetyl transferase, or luciferase) that measure transcriptional read-out. The promoters can be tested as pure DNA, or as DNA bound to chromatin proteins.

In one aspect of the present method, the step of detecting can include detecting the copy number or expression level of one or more genes of the invention in intact animals or tissues obtained from such animals. Mammalian (i.e. mouse, rat, monkey) or non-mammalian (i.e. chicken) species can be the test animals. Sample tissues from a human patient can also be screened. The tissues to be surveyed can be either normal or malignant tissues. The presence and gene copy number or quantity of endogenous mRNA or protein expression of the MYC and/or EIF3H genes can be measured in those tissues. These gene markers can be measured in tissues that are fresh, frozen, fixed or otherwise preserved. They can be measured in cytoplasmic or nuclear organ-, tissue- or cell-extracts; or in cell membranes including but not limited to plasma, cytoplasmic, mitochondrial, golgi or nuclear membranes; in the nuclear matrix; or in cellular organelles and their extracts including but not limited to ribosomes, nuclei, nucleoli, mitochondria, or golgi. Assays for the copy number or endogenous expression of mRNAs or proteins encoded by the MYC and/or EIF3H genes can be performed as described above. Alternatively, intact transgenic animals can be generated for screening for research or validation purposes.

The values obtained from the test and/or control samples are statistically processed using any suitable method of statistical analysis to establish a suitable baseline level using methods standard in the art for establishing such values. Statistical significance according to the present invention should be at least p<0.05.

It will be appreciated by those of skill in the art that differences between the copy number or expression of the MYC and/or EIF3H genes in sensitive versus resistant cells may be small or large. Some small differences may be very reproducible and therefore nonetheless useful. For other purposes, large differences may be desirable for ease of detection of the activity. It will be therefore appreciated that the exact boundary between what is called a positive result and a negative result can shift, depending on the goal of the screening assay. For some assays it may be useful to set threshold levels of change. One of skill in the art can readily determine the criteria for screening of cells given the information provided herein.

The presence and quantity of each gene marker can be measured in primary tumors, metastatic tumors, locally recurring tumors, ductal carcinomas in situ, or other tumors. The markers can be measured in solid tumors that are fresh, frozen, fixed or otherwise preserved. They can be measured in cytoplasmic or nuclear tumor extracts; or in tumor membranes including but not limited to plasma, mitochondrial, golgi or nuclear membranes; in the nuclear matrix; or in tumor cell organelles and their extracts including but not limited to ribosomes, nuclei, mitochondria, golgi.

The copy number and/or level of expression of the MYC and/or EIF3H genes detected in a test or patient sample is compared to a baseline or control level of expression of that gene. More specifically, according to the present invention, a “baseline level” is a control level of biomarker expression against which a test level of biomarker expression (i.e., in the test sample) can be compared. In the present invention, the control level of biomarker expression can be the expression level of the MYC and/or EIF3H genes in a control cell that is sensitive to the EGFR inhibitor, and/or the expression level of the MYC and/or EIF3H genes in a control cell that is resistant to the EGFR inhibitor. Other controls may also be included in the assay. In one embodiment, the control is established in an autologous control sample obtained from the patient. The autologous control sample can be a sample of isolated cells, a tissue sample or a bodily fluid sample, and is preferably a cell sample or tissue sample. According to the present invention, and as used in the art, the term “autologous” means that the sample is obtained from the same patient from which the sample to be evaluated is obtained. The control sample should be of or from the same cell type and preferably, the control sample is obtained from the same organ, tissue or bodily fluid as the sample to be evaluated, such that the control sample serves as the best possible baseline for the sample to be evaluated. In one embodiment, control expression levels of the gene or genes that has been correlated with sensitivity and/or resistance to the EGFR inhibitor has been predetermined, such as in Table 1. Such a form of stored information can include, for example, but is not limited to, a reference chart, listing or electronic file of gene copy numbers or expression levels and profiles for EGFR inhibitor-sensitive and/or EGFR inhibitor-resistant biomarker expression, or any other source of data regarding baseline biomarker expression that is useful in the method of the invention. Therefore, it can be determined, based on the control or baseline level of biomarker expression or biological activity, whether the expression level of a gene or genes in a patient sample is/are more statistically significantly similar to the baseline for EGFR resistance or EGFR sensitivity.

A profile of the copy number or expression levels of the MYC and/or EIF3H genes, including a matrix of both markers, can be generated by one or more of the methods described above. According to the present invention, a profile of the MYC and/or EIF3H genes in a tissue sample refers to a reporting of the copy number or expression level of one of the MYC and EIF3H genes, and includes a classification of the gene with regard to how the gene is regulated in an EGFR inhibitor-sensitive versus an EGFR inhibitor-resistant cells. The data can be reported as raw data, and/or statistically analyzed by any of a variety of methods, and/or combined with any other prognostic marker(s).

Another embodiment of the present invention relates to a plurality of polynucleotides for the detection of the expression of genes as described herein. The plurality of polynucleotides consists of polynucleotides that are complementary to RNA transcripts, or nucleotides derived therefrom, of MYC and/or EIF3H genes and is therefore distinguished from previously known nucleic acid arrays and primer sets. The plurality of polynucleotides within the above-limitation includes at least two or more polynucleotides that are complementary to RNA transcripts, or nucleotides derived therefrom, of one or more of MYC and/or EIF3H genes. Preferably, the plurality of polynucleotides is capable of detecting the copy number or the expression of both MYC and/or EIF3H genes.

In one embodiment, it is contemplated that additional genes that are not regulated differently in an EGFR inhibitor-sensitive versus an EGFR inhibitor-resistant cells can be added to the plurality of polynucleotides. Such genes would not be random genes, or large groups of unselected human genes, as are commercially available now, but rather, would be specifically selected to complement the MYC and/or EIF3H genes. For example, one of skill in the art may wish to add to the above-described plurality of genes one or more genes that are of relevance because they are expressed by a particular tissue of interest (e.g., lung tissue), are associated with a particular disease or condition of interest (e.g., NSCLC), or are associated with a particular cell, tissue or body function (e.g., angiogenesis). The development of additional pluralities of polynucleotides (and antibodies, as disclosed below), which include both the above-described plurality and such additional selected polynucleotides, are explicitly contemplated by the present invention.

According to the present invention, a plurality of polynucleotides refers to at least 2, and more preferably at least 3, and more preferably at least 4, and more preferably at least 5, and more preferably at least 6, and more preferably at least 7, and more preferably at least 8, and more preferably at least 9, and more preferably at least 10, and so on, in increments of one, up to any suitable number of polynucleotides, including at least 100, 500, 1000, 10⁴, 10⁵, or at least 10⁶ or more polynucleotides.

In one embodiment, the polynucleotide probes are conjugated to detectable markers. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads, fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Preferably, the polynucleotide probes are immobilized on a substrate.

In one embodiment, the polynucleotide probes are hybridizable array elements in a microarray or high density array. Nucleic acid arrays are well known in the art and are described for use in comparing expression levels of particular genes of interest, for example, in U.S. Pat. No. 6,177,248, which is incorporated herein by reference in its entirety. Nucleic acid arrays are suitable for quantifying a small variations in expression levels of a gene in the presence of a large population of heterogeneous nucleic acids. Knowing the identity of the genes of the present invention, nucleic acid arrays can be fabricated either by de novo synthesis on a substrate or by spotting or transporting nucleic acid sequences onto specific locations of substrate. Nucleic acids are purified and/or isolated from biological materials, such as a bacterial plasmid containing a cloned segment of sequence of interest. It is noted that all of the genes identified by the present invention have been previously sequenced, at least in part, such that oligonucleotides suitable for the identification of such nucleic acids can be produced. The database accession number for each of the genes identified by the present inventors is provided in Table 1. Suitable nucleic acids are also produced by amplification of template, such as by polymerase chain reaction or in vitro transcription. Synthesized oligonucleotide arrays are particularly preferred for this aspect of the invention. Oligonucleotide arrays have numerous advantages, as opposed to other methods, such as efficiency of production, reduced intra- and inter array variability, increased information content and high signal-to-noise ratio.

One of skill in the art will appreciate that an enormous number of array designs are suitable for the practice of this invention. An array will typically include a number of probes that specifically hybridize to MYC and/or EIF3H gene sequences. In addition, in a preferred embodiment, the array will include one or more control probes. The high-density array chip includes “test probes.” Test probes could be oligonucleotides that range from about 5 to about 45 or 5 to about 500 nucleotides (including any whole number increment in between), more preferably from about 10 to about 40 nucleotides and most preferably from about 15 to about 40 nucleotides in length. In other particularly preferred embodiments the probes are 20 or 25 nucleotides in length. In another preferred embodiments, test probes are double or single strand DNA sequences. DNA sequences are isolated or cloned from natural sources or amplified from natural sources using natural nucleic acids as templates, or produced synthetically. These probes have sequences complementary to particular subsequences of the genes whose expression they are designed to detect. Thus, the test probes are capable of specifically hybridizing to the target nucleic acid they are to detect.

Another embodiment of the present invention relates to a plurality of antibodies, or antigen binding fragments thereof, for the detection of the expression of MYC and/or EIF3H genes according to the methods of the present invention. The plurality of antibodies, or antigen binding fragments thereof, consists of antibodies, or antigen binding fragments thereof, that selectively bind to proteins encoded by MYC and/or EIF3H genes. According to the present invention, a plurality of antibodies, or antigen binding fragments thereof, refers to at least 2, and more preferably at least 3, and more preferably at least 4, and more preferably at least 5, and more preferably at least 6, and more preferably at least 7, and more preferably at least 8, and more preferably at least 9, and more preferably at least 10, and so on, in increments of one, up to any suitable number of antibodies, or antigen binding fragments thereof, including at least 100, 500, or at least 1000 antibodies, or antigen binding fragments thereof.

The invention also extends to non-antibody polypeptides, sometimes referred to as binding partners or antigen binding peptides, that have been designed to bind specifically to, and either activate or inhibit as appropriate, a target protein. Examples of the design of such polypeptides, which possess a prescribed ligand specificity are given in Beste et al. (Proc. Natl. Acad. Sci. 96:1898-1903, 1999), incorporated herein by reference in its entirety.

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

Finally, the MYC and/or EIF3H genes, or their RNA or protein products, can serve as targets for therapeutic strategies. For example, neutralizing antibodies could be directed against one of the protein products of a selected gene, expressed on the surface of a tumor cell. Alternatively, regulatory compounds that regulate (e.g., upregulate or downregulate) the expression and/or biological activity of MYC and/or EIF3H genes can be identified and/or designed using the methods described herein. For example, in one aspect, a method of using the MYC and/or EIF3H genes as a target includes the steps of: (a) contacting a test compound with a cell that expresses MYC and/or EIF3H genes; and (b) identifying compounds, wherein the compounds can include: (i) compounds that increase the expression or activity of the MYC and/or EIF3H genes, or the proteins encoded thereby, that are correlated with sensitivity to an EGFR inhibitor; and (ii) compounds that decrease the expression or activity of MYC and/or EIF3H genes, or the proteins encoded thereby, that are correlated with resistance to an EGFR inhibitor. The compounds are thereby identified as having the potential to enhance the efficacy of EGFR inhibitors.

The period of contact with the compound being tested can be varied depending on the result being measured, and can be determined by one of skill in the art. As used herein, the term “contact period” refers to the time period during which cells are in contact with the compound being tested. The term “incubation period” refers to the entire time during which cells are allowed to grow prior to evaluation, and can be inclusive of the contact period. Thus, the incubation period includes all of the contact period and may include a further time period during which the compound being tested is not present but during which expression of genes is allowed to continue prior to scoring. Methods to evaluate gene expression in a cell according to the present invention have been described previously herein.

If a suitable therapeutic compound is identified using the methods and genes of the present invention, a composition can be formulated. A composition, and particularly a therapeutic composition, of the present invention generally includes the therapeutic compound and a carrier, and preferably, a pharmaceutically acceptable carrier. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site is preferably a tumor cell. In some embodiments, a suitable site for delivery is a site of inflammation, near the site of a tumor, or a site of any other disease or condition in which regulation of the genes identified herein can be beneficial. Preferred pharmaceutically acceptable carriers are capable of maintaining a compound, a protein, a peptide, nucleic acid molecule or mimetic (drug) according to the present invention in a form that, upon arrival of the compound, protein, peptide, nucleic acid molecule or mimetic at the cell target in a culture or in patient, the compound, protein, peptide, nucleic acid molecule or mimetic is capable of interacting with its target.

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

One type of pharmaceutically acceptable carrier includes a controlled release formulation that is capable of slowly releasing a composition of the present invention into a patient or culture. As used herein, a controlled release formulation comprises a compound of the present invention (e.g., a protein (including homologues), a drug, an antibody, a nucleic acid molecule, or a mimetic) in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a patient, form a solid or a gel in situ. Preferred carriers are also biodegradable (i.e., bioerodible). When the compound is a recombinant nucleic acid molecule, suitable delivery vehicles include, but are not limited to liposomes, viral vectors or other delivery vehicles, including ribozymes. Natural lipid-containing delivery vehicles include cells and cellular membranes. Artificial lipid-containing delivery vehicles include liposomes and micelles. A delivery vehicle of the present invention can be modified to target to a particular site in a patient, thereby targeting and making use of a compound of the present invention at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

A pharmaceutically acceptable carrier, which is capable of targeting is herein referred to as a “delivery vehicle.” Delivery vehicles of the present invention are capable of delivering a composition of the present invention to a target site in a patient. A “target site” refers to a site in a patient to which one desires to deliver a composition. For example, a target site can be any cell, which is targeted by direct injection or delivery using liposomes, viral vectors or other delivery vehicles, including ribozymes and antibodies. Examples of delivery vehicles include, but are not limited to, artificial and natural lipid-containing delivery vehicles, viral vectors, and ribozymes. Natural lipid-containing delivery vehicles include cells and cellular membranes. Artificial lipid-containing delivery vehicles include liposomes and micelles. A delivery vehicle of the present invention can be modified to target to a particular site in a subject, thereby targeting and making use of a compound of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically, targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

Another preferred delivery vehicle comprises a viral vector. A viral vector includes an isolated nucleic acid molecule useful in the present invention, in which the nucleic acid molecules are packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

A composition can be delivered to a cell culture or patient by any suitable method. Selection of such a method will vary with the type of compound being administered or delivered (i.e., compound, protein, peptide, nucleic acid molecule, or mimetic), the mode of delivery (i.e., in vitro, in vivo, ex vivo) and the goal to be achieved by administration/delivery of the compound or composition. According to the present invention, an effective administration protocol (i.e., administering a composition in an effective manner) comprises suitable dose parameters and modes of administration that result in delivery of a composition to a desired site (i.e., to a desired cell) and/or in the desired regulatory event.

Administration routes include in vivo, in vitro and ex vivo routes. In vivo routes include, but are not limited to, oral, nasal, intratracheal injection, inhaled, transdermal, rectal, and parenteral routes. Preferred parenteral routes can include, but are not limited to, subcutaneous, intradermal, intravenous, intramuscular and intraperitoneal routes. Intravenous, intraperitoneal, intradermal, subcutaneous and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art. Direct injection techniques are particularly useful for suppressing graft rejection by, for example, injecting the composition into the transplanted tissue, or for site-specific administration of a compound, such as at the site of a tumor. Ex vivo refers to performing part of the regulatory step outside of the patient, such as by transfecting a population of cells removed from a patient with a recombinant molecule comprising a nucleic acid sequence encoding a protein according to the present invention under conditions such that the recombinant molecule is subsequently expressed by the transfected cell, and returning the transfected cells to the patient. In vitro and ex vivo routes of administration of a composition to a culture of host cells can be accomplished by a method including, but not limited to, transfection, transformation, electroporation, microinjection, lipofection, adsorption, protoplast fusion, use of protein carrying agents, use of ion carrying agents, use of detergents for cell permeabilization, and simply mixing (e.g., combining) a compound in culture with a target cell.

In the method of the present invention, a therapeutic compound, as well as compositions comprising such compounds, can be administered to any organism, and particularly, to any member of the Vertebrate class, Mammalia, including, without limitation, primates, rodents, livestock and domestic pets. Livestock include mammals to be consumed or that produce useful products (e.g., sheep for wool production). Preferred mammals to protect include humans. Typically, it is desirable to obtain a therapeutic benefit in a patient. A therapeutic benefit is not necessarily a cure for a particular disease or condition, but rather, preferably encompasses a result which can include alleviation of the disease or condition, elimination of the disease or condition, reduction of a symptom associated with the disease or condition, prevention or alleviation of a secondary disease or condition resulting from the occurrence of a primary disease or condition, and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or causes. As such, to protect a patient from a disease includes both preventing disease occurrence (prophylactic treatment) and treating a patient that has a disease (therapeutic treatment) to reduce the symptoms of the disease. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

Another embodiment of the invention relates to the use of any of the therapeutic compounds, proteins or compositions described above in the preparation of a medicament for the treatment of cancer.

Each publication or patent cited herein is incorporated herein by reference in its entirety.

Various aspects of the invention are described in the following examples; however, the following examples are provided for the purpose of illustration and are not intended to limit the scope of the present invention.

### EXAMPLE

The following example describes the validation of MYC and EIF3H co-amplification in NSCLC with increased sensitivity to EGFR inhibitor therapy.

In this study the authors investigated if EIF3H was amplified, and whether MYC and/or EIF3H genomic gain affected response to EGFR tyrosine kinase inhibitors in NSCLC.

Metastatic NSCLC patients (N=54) treated with gefitinib were analyzed for EIF3H and MYC genes by FISH, using a custom-designed 3-color DNA probe set.

Results: Amplification of EIF3H (ratio EIF3H/CEP8>2), was observed in 10 cases (18.5%), and MYC was co-amplified in all. MYC amplification without co-amplification of EIF3H was observed in 2 cases (3.7%). Response to gefitinib therapy was higher in MYC amplified than in non-amplified patients (25% versus 14%, p=0.4) and in EIF3H amplified versus non amplified (30% versus 14%, p=0.3). In order to investigate whether this trend for higher response was due to chance or reflected a significant biological difference, a Receiver Operating Characteristic (ROC) analysis was conducted to identify the cut-off for MYC and EIF3H copy number that best discriminated sensitive and resistant patient populations. MYC FISH positive patients (mean ≧2.79) had significantly higher response rate (RR: 31% versus 0%, p=0.003), significantly longer time to progression (TTP: 4.4 versus 2.6 months, p=0.01) and survival (OS:13.8 versus 6.4 months, p=0.02) than MYC FISH negative patients (mean <2.79). EIF3H FISH positive patients (mean ≧2.75) had significantly higher RR (32% versus 0%, p=0.002), significantly longer TTP (4.4 versus 2.7 months, p=0.01) and OS (17.8 versus 6.4 months, p=0.01) than EIF3H FISH negative patients (mean <2.75).

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1. A method to select a cancer patient who is predicted to benefit from therapeutic administration of an EGFR inhibitor, an agonist thereof, or a drug having substantially similar biological activity as EGFR inhibitor, comprising:
a) providing a sample of tumor cells from a patient to be tested;
b) detecting in the sample the copy number of genes chosen from a panel of genes whose copy number has been correlated with sensitivity to an EGFR inhibitor;
c) detecting in the sample the copy number of genes chosen from a panel of genes whose copy number has been correlated with resistance to an EGFR inhibitor;
d) comparing the level of copy number of the genes detected in the patient sample to the copy number of the genes that have been correlated with sensitivity to the EGFR inhibitor;
e) comparing the copy number of the genes detected in the patient sample to the copy number of the genes that have been correlated with resistance to the EGFR inhibitor; and
f) selecting a patient as predicted to benefit from therapeutic administration of the EGFR inhibitor, if the copy number of the genes in the patient's tumor cells is statistically more similar to the copy number of the genes that have been correlated with sensitivity to the EGFR inhibitor than to resistance to the EGFR inhibitor.

2. The method of claim 1, wherein the panel of genes is identified by a method comprising:
a) providing a sample of tumor cells that are sensitive to treatment with the EGFR inhibitor;
b) providing a sample of tumor cells that are resistant to treatment with the EGFR inhibitor;
c) detecting the copy number of at least one gene in the EGFR inhibitor-sensitive cells as compared to the copy number of at least one gene in the EGFR inhibitor-resistant cells; and
d) identifying genes having a copy number in EGFR inhibitor-sensitive cells that are statistically significantly different than the copy number of the genes in EGFR inhibitor-resistant cells, as potentially being a molecule that interacts with the EGFR pathway to enhance responsiveness to EGFR inhibitors.

3. (canceled)

4. (canceled)

5. The method of claim 1, wherein steps (b) and (c) comprise detecting in the sample the copy number of at least one of MYC and EIF3H genes;
wherein steps (d) and (e) comprise comparing the copy number of the genes detected in the patient sample to a copy number of the genes that have been correlated with sensitivity to gefitinib and to resistance of gefitinib; and,
wherein step (f) comprises selecting the patient as being predicted to benefit from therapeutic administration of gefitinib, an agonist thereof, and a drug having substantially similar biological activity as gefitinib, if the copy number of the genes in the patient's tumor cells is statistically more similar to the copy number of the genes that have been correlated with sensitivity to gefitinib than to resistance to gefitinib.

6. (canceled)

7. (canceled)

8. (canceled)

9. (canceled)

10. The method of claim 1, wherein the copy number of the genes is detected by hybridization of one of a portion of the gene and a transcript thereof, to a nucleic acid molecule comprising one of a portion of the gene and a transcript thereof, conjugated to a detectable marker.

11. (canceled)

12. (canceled)

13. The method of claim 1, comprising comparing the copy number of the genes to the copy number of the genes in a cell from a non-cancerous cell of the same type.

14. The method of claim 1, comprising comparing the copy number of the genes to the copy number of the genes in an autologous, non-cancerous cell from the patient.

15. The method of claim 1, comprising comparing the copy number of the genes to the copy number of the genes in a control cell that is resistant to an EGFR inhibitor.

16. The method of claim 1, comprising comparing the copy number of the genes to the copy number of the genes in a control cell that are sensitive to an EGFR inhibitor.

17. (canceled)

18. (canceled)

19. A method to select a cancer patient who is predicted to benefit from therapeutic administration of an EGFR inhibitor, an agonist thereof, and a drug having substantially similar biological activity as an EGFR inhibitor, comprising:
a) providing a sample of tumor cells from a patient to be tested;
b) detecting in the sample the expression of genes chosen from a panel of genes whose expression has been correlated with sensitivity to an EGFR inhibitor;
c) detecting in the sample the expression of genes chosen from a panel of genes whose expression has been correlated with resistance to an EGFR inhibitor;
d) comparing the level of expression of the genes detected in the patient sample to a level of expression of the genes that have been correlated with sensitivity and resistance to the EGFR inhibitor; and
e) selecting the patient as being predicted to benefit from therapeutic administration of the EGFR inhibitor, if the expression of the genes in the patient's tumor cells is statistically more similar to the expression levels of the genes that have been correlated with sensitivity to the EGFR inhibitor than to resistance to the EGFR inhibitor.

20. The method of claim 19, wherein the panel of genes in steps (b) and (c) are identified by a method comprising:
a) providing a sample of cells that are sensitive to treatment with the EGFR inhibitor;
b) providing a sample of cells that are resistant to treatment with the EGFR inhibitor;
c) detecting the expression of at least one gene in the EGFR inhibitor-sensitive cells as compared to the level of expression of at least one gene in the EGFR inhibitor-resistant cells; and
d) identifying genes having a level of expression in EGFR inhibitor-sensitive cells that is statistically significantly different than the level of expression of the genes in EGFR inhibitor-resistant cells, as potentially being a molecule that interacts with the EGFR pathway to enhance responsiveness to EGFR inhibitors.

21. (canceled)

22. (canceled)

23. The method of claim 19, wherein steps (b) and (c) comprise detecting in the sample the expression of at least one of MYC and EIF3H genes;
wherein step (d) comprises comparing the level of expression of the genes detected in the patient sample to a level of expression of the genes that have been correlated with sensitivity and resistance to gefitinib; and,
wherein step (e) comprises selecting the patient as being predicted to benefit from therapeutic administration of gefitinib, an agonist thereof, and a drug having substantially similar biological activity as gefitinib, if the expression of the genes in the patient's tumor cells is statistically more similar to the expression levels of the genes that have been correlated with sensitivity to gefitinib than to resistance to gefitinib.

24. (canceled)

25. (canceled)

26. (canceled)

27. (canceled)

28. The method of claim 19, wherein expression of the genes is detected by detecting hybridization of at least a portion of the gene or a transcript thereof, to a nucleic acid molecule comprising a portion of the gene and a transcript thereof in a nucleic acid array.

29. The method of claim 19, wherein expression of the genes are detected by detecting the production of proteins encoded by the genes.

30. The method of claim 19, comprising comparing the expression of the genes to the expression of the genes in a cell from a non-cancerous cell of the same type.

31. The method of claim 19, comprising comparing the expression of the genes to the expression of the genes in an autologous, non-cancerous cell from the patient.

32. The method of claim 19, comprising comparing the expression of the genes to the expression of the genes in a control cell that is resistant to the EGFR inhibitor.

33. The method of claim 19, comprising comparing the expression of the genes to the expression of the genes in a control cell that is sensitive to the EGFR inhibitor.

34. (canceled)

35. (canceled)

36. (canceled)

37. A plurality of polynucleotides for the detection of the copy number of genes that are selected from the group consisting of EGFR inhibitor-sensitive genes, EGFR inhibitor-resistant genes, agonists thereof, and drugs having substantially similar biological activity as EGFR inhibitors;
wherein the plurality of polynucleotides consists of at least two polynucleotides, wherein each polynucleotide is at least 5 nucleotides in length, and wherein each polynucleotide is selected from the group consisting of polynucleotides that are complementary to a genomic sequence, an RNA transcript, and nucleotides derived therefrom, of a gene that has a gene copy number that is different in EGFR inhibitor-sensitive tumor cells as compared to EGFR inhibitor-resistant cells.

38. (canceled)

39. (canceled)

40. (canceled)

41. (canceled)

42. The plurality of polynucleotides of claim 37, wherein said polynucleotide probes are hybridizable array elements in a microarray.

43. (canceled)

44. (canceled)

45. (canceled)

46. (canceled)

47. (canceled)

48. (canceled)

49. (canceled)

50. (canceled)

51. (canceled)
